(19) United States
(12) Patent Application Publication
(10) Pub. No.: US 2003/0124142 A1
(43) Pub. Date: Jul. 3, 2003

(54) Immunomodulatory constructs and their uses

(76) Inventors: John David Fraser, Meadowbank (NZ); Melissa Joy Nicholson, Cambridge, MA (US)

Correspondence Address: Y. ROCKY TSAO, Fish & Richardson P.C., 225 Franklin Street, Boston, MA, 02110-2804, US

(21) Appl. No.: 10/006,797

(22) Filed: Dec. 4, 2001

Related U.S. Application Data
(60) Provisional application No. 60/251,243, filed on Dec. 4, 2000.

(30) Foreign Application Priority Data
Dec. 4, 2001 (WO) PCT/NZ01/00269

(51) Int. Cl. A61K 39/02; C07K 14/31; C07K 14/315

(52) U.S. Cl. 424/190.1; 530/350

(57) ABSTRACT

An immunomodulator which includes an antigen-presenting-cell (APC) targeting molecule coupled to an immunomodulatory antigen, wherein the APC-targeting molecule mimics a superantigen but does not include a fully functional T-cell receptor binding site. Also disclosed are a method of therapeutic or prophylactic treatment of a disorder, including administrating to a subject in need thereof a pharmaceutical composition or a vaccine containing the immunomodulator; use of the immunomodulator for the preparation of a medicament for the therapeutic or prophylactic treatment of a disorder; and a method of preparing the immunomodulator.

### TECHNICAL FIELD

This invention relates to immunomodulatory constructs and their use. In particular, it relates to constructs which target antigen-presenting-cells for the purpose of enhancing or suppressing a host immune response, and to methods of enhancing antigenicity of compounds.

### BACKGROUND ART

Professional antigen-presenting-cells (APC) are essential to initiate a primary immune response in a non-immune, naive animal. The most important APC is the Dendritic Cell (DC), which is found as an interdigitating cell at all regions of the body, at an interface with the environment (i.e. skin and mucosal surfaces such as the lung, airways, nasal passage etc). Antigens presented by DCs are profoundly immunogenic. One important phenotypic marker of the DC is a very high level of surface MHC class II expression. Activated DCs migrate to secondary lymph nodes to “prime” both CD4 and CD8 T cells which proceed as antigen activated effector cells, to proliferate, produce cytokines and regulate the humoral response of B-lymphocytes. Thus, antigen presentation by DC appears to be the obligate first step in any adaptive immune response. Other APCs such as macrophages and B-cells appear to be important in later, secondary responses and by themselves are not effective in the initial priming of a response. Thus the DC is generally regarded as the most important cell to target for enhancement of immune responses.

The targeting of antigens to DC can however be problematic. For example, many peptides by themselves are poorly antigenic and immunogenic because they are not efficiently delivered to APC in vivo. They are equally not taken up by APC very efficiently and do not elicit the second signals required for efficient antigen presentation.

Superantigens are a family of semi-conserved bacterial proteins that target the immune system by binding simultaneously to the T cell Receptor (TcR) via the Vβ domain on T lymphocytes and MHC class II molecules expressed on APC including dendritic cells.

Superantigens (SAgs) are the most potent immune mitogens known and activate large numbers of T cells at femto-attomolar concentrations (10⁻¹⁵-10⁻¹⁸M). They cause significant toxicity due to the massive systemic cytokine release by T cells. There are currently 19 members of the staphylococcal and streptococcal superantigen family.

Terman (WO 98/26747) discloses therapeutic compositions employing superantigens. It is suggested that superantigens, in conjunction with one or more additional immunotherapeutic antigens, may be used to either induce a therapeutic immune response directed against a target or to inhibit a disease-causing immune response. Terman further describes the formation of immunotherapeutic antigen-superantigen polymers. Such polymers include those where the superantigen component is coupled to a peptide antigen by a secondary amine linkage. However, there is no teaching or suggestion by Terman that the superantigen component be one from which the TcR binding function has been wholly or partly ablated. Indeed, there is no recognition that a TcR binding is not essential to activation of APCs and to stimulation of an immune response against the antigenic component of the polymer.

Thus, wild-type SAgs, or modified SAgs which retain the ability to bind to TcR, are of little use because they themselves elicit massive, indiscriminate T cell responses by binding to the TcR. This TcR cross-linking appears to be the major cause of their toxicity ¹².

There exists a need therefore for improved immunomodulators which exploit the unique features of DC targeting and activation of SAgs to deliver and enhance the T cell recognition of antigens such as peptides that are normally non-immunogenic or have low immunogenicity, yet are efficacious and have low toxicity.

It is an object of the present invention to overcome or ameliorate at least some of the disadvantages of the prior art, or to provide a useful alternative.

### SUMMARY OF THE INVENTION

According to a first aspect there is provided an immunomodulator which comprises an antigen-presenting-cell (APC) targeting molecule coupled to an immunomodulatory antigen, wherein said APC-targeting molecule mimics a superantigen but does not include a fully functional T-cell receptor binding site.

According to a second aspect there is provided an immunomodulator which comprises an antigen-presenting cell (APC) targeting molecule coupled to an immunomodulatory antigen, wherein said APC-targeting molecule is a molecule which is structurally a superantigen but for a disrupted T-cell receptor binding site such that the molecule has little or no ability to activate T-cells.

Preferably the T-cell receptor binding site, or at least part thereof, of the antigen-presenting-cell (APC) targeting molecule is derived from Staphylococcus aureus and/or Streptococcus pyogenes. Particularly preferred is a targeting molecule derived from SPE-C and the preferred truncation involves deletion of residues 22-90 from the wild-type SPE-C sequence. However it will be clear to those skilled in the art that other SAgs which have a similar or otherwise known TcR binding region of the molecule may also be advantageously used, for example SMEZ, SEA and the like.

The T-cell receptor binding site, or at least a part thereof, of the antigen-presenting-cell (APC) targeting molecule can also been modified by substitution or addition, to remove or minimise TcR binding. An example of such a targeting molecule is SPEC-Y15A R181Q of the present invention.

A particularly preferred intermediate in the generation of the immunomodulator is Y15A.C27S.N79C.

Preferably the coupling between the antigen-presenting-cell (APC) targeting molecule and the immunomodulatory antigen will be reversible. However, it will be understood from the following description that what is preferably required is that the antigen-presenting-cell (APC) targeting molecule is capable of releasing the immunomodulatory antigen so that it is correctly presented by the APC. Thus, it would also be clear that the release of the immunomodulatory antigen from the immunomodulator may be achieved by intracellular or intralysosomal enzymatic cleavage. This process may be assisted by introducing the appropriate proteolytic site into the coupling region of the immunomodulator. The release may also be achieved by chemical means, which includes redox reactions involving disulphides and free sylphydryl groups. This process may also be assisted by introducing into the coupling region certain amino acid residues, e.g. cysteine.

Preferably the immunomodulatory antigen is a protein, a polypeptide and/or a peptide however similar principles may be applied to antigens which are non-proteinaceous, for example nucleic acids or carbohydrates.

The immunomodulatory antigen may be entirely non-immunogenic when not coupled to the antigen-presenting cell (APC) targeting molecule but the immunomodulators of the present invention may also incorporate antigens which are immunogenic, in order to improve their efficacy. Thus the present invention is equally applicable to for example to new vaccines as it is to those which are already known and used but which can be improved by means of the immunomodulators of the present invention.

According to a third aspect there is provided a pharmaceutical composition comprising an immunomodulator according to the present invention and a pharmaceutically acceptable carrier, adjuvant, excipient and/or solvent.

According to a fourth aspect there is provided a vaccine comprising an immunomodulator according to the present invention.

According to a fifth aspect there is provided a method of therapeutic or prophylactic treatment of a disorder which requires the induction or stimulation of the immune system, comprising the administration to a subject requiring such treatment of an immunomodulator or of a pharmaceutical composition according to the present invention.

Preferably the disorder is selected from the group consisting of bacterial, viral, fungal or parasitic infection, autoimmunity, allergy and/or pre-neoplastic or neoplastic transformation.

According to a fifth aspect there is provided the use of an immunomodulator according to the first or the second aspect for the preparation of a medicament for the therapeutic or prophylactic treatment of a disorder which requires the induction or stimulation of the immune system.

The preferred disorder is selected from the group consisting of bacterial, viral, fungal or parasitic infection, autoimmunity, allergy and/or pre-neoplastic or neoplastic transformation.

According to a sixth aspect there is provided a method of preparing an immunomodulator comprising the steps of:

a introducing a modification and/or a deletion into the T-cell binding site of an antigen-presenting cell (APC) targeting molecule which is structurally a superantigen, and

b coupling thereto and immunomodulatory antigen.

Preferably the antigen-presenting cell (APC) targeting molecule is selected from the group of SPE-C, SMEZ and SEA and more preferred are the antigen-presenting cell (APC) targeting molecules SPE-C Y15A. R181Q or SPEC (-20-90). Even more preferred is SPEC-Y15A.C27S.N79C.R181Q

It will be understood however that more than one antigen-presenting cell (APC) targeting molecule may be employed and that a combination of immunomodulators may be used in any treatment.

### BRIEF DESCRIPTION OF THE FIGURES

FIG. 1. Antigenicity of SAG:PCC conjugate

FIG. 2. Immunogenicity of SPEC:PCC conjugate

FIG. 3. Proliferation of 5C.C7 LN cells to SPEC-CytC vs MHC−/−SPEC-CytC and free CytC peptide in vitro

FIG. 4. Proliferative responses of SMEZ TcR mutants

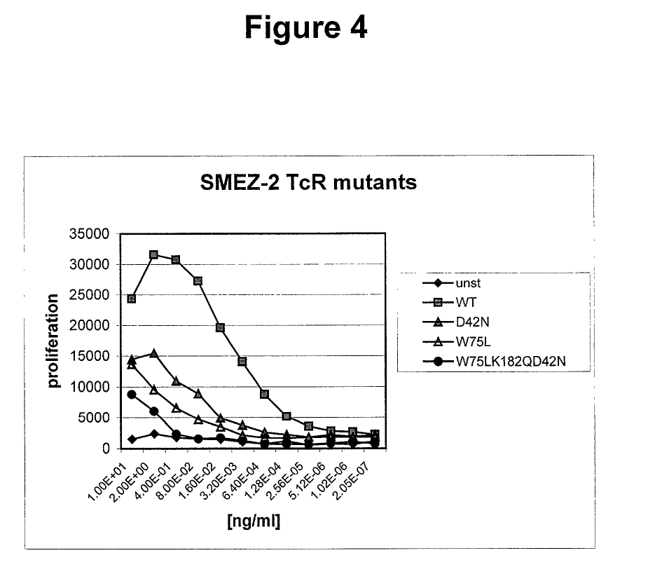

FIG. 5. Proliferative responses of 5C.C7 LN Cells with PCC-SAg Complexes. (Legend: The red line indicates the proliferative response to PCC protein alone. The blue square line shows that the response to PCC-SPEC is 100-fold more antigenic than the unconjugated PCC protein. The green square line is the response to PCC-SMEZ and is approximately 80 fold more antigenic than to unconjugated PCC protein. The black square shows the response to PCC conjugated to SPEC defective in MHC class II binding is no greater than the response to unconjugated PCC protein. The triangles represent the proliferative response of T cells to SAG and PCC together as a mixture but not conjugated).

### DESCRIPTION OF THE PREFERRED EMBODIMENT

The present invention is based at least in part on an unexpected observation that a molecule which mimics a superantigen but which lacks a fully functional TcR binding site can, when coupled to an immunomodulatory antigen, bind and activate APCs to a degree not previously known or suspected. Thus, such immunomodulatory constructs are effective in antigen presentation without the requirement to bind to the TcR. This is of particular relevance to moieties which have low or nonexistent immunogenicity, such as peptides, proteins, nucleic acids, whole viruses etc

The applications of this technology rely on the ability to generate a wide variety of immunomodulatory reagents that combine the delivery capacity and APC activating potential of the TcR ablated superantigens with the specificity of a coupled antigen.

A preferred use of this technique is to enhance responses to synthetic peptides as has been displayed herein with the PCC peptide. However, the antigen need not be a synthetic peptide, but could be a native or recombinant polypeptide, protein of even whole disabled virus. Further, the antigen need not be proteinaceous and may be a nucleic acid or carbohydrate antigen. Also, the present invention can be applied to antigens which are immunogenic, by improving immunogenicity or reducing the quantity of antigen required to induce an immune response

Peptides can be designed to be either stimulatory (i.e. generate agonist responses) or immunosuppressive (i.e. generate antagonist responses) to induce tolerance depending on the primary sequence of the peptide. This is useful in either promoting immunity for vaccination against pathogens such as viruses, bacteria and other micro-organisms, or for generating specific anti-tumour immunity using tumour specific peptides.

Antagonist responses induce T cell tolerance to antigen and might be useful to suppressing unwanted autoimmune reaction to self-antigens e.g. proteins and/or nucleic acids, in the case of diseases such as multiple sclerosis, diabetes or rheumatoid arthritis.

Many autoimmune diseases have their basis in an auto-reactive T cell response to self antigens. Diseases such as rheumatoid arthritis, multiple sclerosis and diabetes mellitus are such examples.

The present invention will now be exemplified more particularly with reference to non-limiting examples.

### EXAMPLES

### Example 1

### Cloning and Expression of Superantigen Genes

Genes coding for individual wild-type superantigens were isolated and cloned directly from the DNA of isolates of Staphylococcus aureus or Streptococcus pyogenes using polymerase chain reaction (PCR) and oligonucleotides inferred from published sequences. All wild type sequences have been confirmed by DNA sequencing.

The methods used for isolation, cloning and sequencing are standard laboratory procedures and are described in for example Goshorn S. C. , Schlievert P. M. 1988. Nucleotide sequence of streptococcal pyrogenic exotoxin type C. Infect Immun. 56(9):2518-20. Proft T, Moffatt S L, Berkahn C J, Fraser J D. 1999. Identification and characterization of novel superantigens from Streptococcus pyogenes. J Exp Med. January 4; 189(1):89-102, all incorporated herein by reference.

A summary of the SPE-C single domain molecule and its derivation is set out below, including the comparative proliferative response of human T cells.

### C-terminal Single Domain of SPE-C

(C-terminal single domain references the term “truncated SPE-C” and is a reference to the explicitly stated SPEC-(-20-90). The parenthesized numbers represent that part of the native SPE-C that has been deleted as outlined in the procedure below)

Vector: pGEX-3C (variation of pGEX-2T)

Host: DH5□

Antibiotic resistance: Ampicillin

Restriction sites: 5′ BamH1, 3′ EcoR1

Brief Expression protocol:

Grow overnight culture in LB-Amp at 37° C. with shaking.

Dilute overnight culture 1:10 with pre-warmed LB-Amp.

Grow for another hour or until the absorbance at 600 nm is 0.9.

Cool culture to 30° C.

Induce protein expression with 0.1 mM IPTG.

Incubate at 30° C. with shaking for 4-5 hours.

Harvest cells and resuspend in 10 mls of GSH Buffer 1 (25 mM

Tris.Cl pH 7.4/50 mM NaCl/1 mM EDTA) for every 1 gram of pellet.

Sonicate to lyse cells and release soluble fusion protein.

Spin lysate to remove insoluble material.

Dialyse lysate overnight in GSH Buffer 1 to remove endogenous GSH (this step will increase yields but is not essential).

Purify GST-Fusion protein from bacterial proteins using GSH agarose affinity chromatography.

Cut purified fusion protein overnight with 3C protease at 4° C. (NB to add DTT)

Dialyse cut fusion protein into 10 mM PO₄ pH 6.0 overnight.

Purify C-terminal Single Domain from GST using cation exchange chromatography (ie MonoS column—elute with pH gradient 6.0-7.0 over 20 column volumes)

Sequence Details

Includes residues 1-21 of SPE-C, 4 amino acid linker which is the Factor X protease cleavage site, and then residues 91-208 of SPE-C.

[image]

Protein Parameters

[image]

Molecular Weight: 16543

Theoretical pI: 7.02

Theoretical Extinction data (6M Guanidine-HCl/20 mM phosphate, pH 6.5)

Assuming all cysteines are reduced:

Molar A280: 8960

A280/cm (1 mg/ml): 0.542

### Activity of C-terminal Domain SPE-C

PBL Stimulation Assay

Peripheral blood lymphocytes are isolated from blood using Hypaque-Ficoll. A 5 fold serial dilution of toxin in RPMI (complete) is set up in a 96 well plate. 1×10⁵ PBLs is added to each well containing varying concentrations of toxins. The plates are left to incubate for 3 days after which time [³H]Thymidine is added to each well to measure proliferation. The cells are harvested the next day and [³H]Thymidine incorporation is measured.

[image]

The above figure shows that the C-terminal domain SPE-C does not have stimulatory activity above background with human PBLs. This is most likely due to the fact that it cannot interact with the TcR on T cells or cross-link MHC on the cell surface of antigen presenting cells.

### Example 2

### Ablation of TcR Binding Residues in Superantigens

The gene from SPE-C was derived from a patient isolate of Streptococcus pyogenes by PCR using synthetic primers to the 5′ and 3′ end of the genes. These primer sequences were obtained from the published sequence of Goshorn S C, Schlievert P M. 1988. Nucleotide sequence of streptococcal pyrogenic exotoxin type C. Infect Immun. 56(9):2518-20. GenBank accession number M35514. Any other Streptococcus pyogenese isolate can also be used for this purpose.

Primers used to amplify the SPEC gene are listed in table 1 as SPEC-N-terminal and SPEC-C-terminal. The sequence was confirmed by DNA sequencing.

The full length SPE-C gene was sub-cloned into the expression vector pGEX-3T (Pharmacia) following manufacturers instructions which was used to transform the bacteria E. coli using standard procedures (Maniatis et al, ). Recombinant SPE-C fused to glutathione-S-transferase was purified from E. coli cultures using Glutathione Agarose affinity chromatography.

TABLE 1

Primers used for amplification of the SPEC gene and introduction of mutations or
truncations

| | | |
|---|---|---|
| SPEC-N-terminal | CGGGATCCGACTCTCAAGAAAGACA | |
| SPEC-C-terminal | CTGAATTCTTATTTTTCAAGAT | |
| SPEC-Y15A | GATTTACTTTGTGCATACAC | GTGTATGCACAAAGTAAATC |
| SPEC-N79C | ATATTCTTTGTTCTCACA | TATAAGAAACAAGAGTGT |
| SPEC-Y15C | GATTTACTTTGTGCATACAC | GTGTATGCACAAAGTAAATC |
| SPEC-R181Q | GAAGGGACTCAATCAGATATTTTTGC | GACAAAATATCTGATTGAGTCCCTTC |
| SPEC-(-20-90) | ATCGAAGGTCGTACGCCTGCTCAAAATAATAAAG | ACGACCTTCGATAGGAGTTATAGTGTAT |
| SPEC-C27S | GATTATAAAGATTCCAGGGTAA | TTACCCTGGAATCTTTATAATC |

Sequential introduction of current mutations into SPE-C.

1. SPEC-C27S

To remove a naturally occurring cysteine that interferes with the coupling of antigen to the preferred site at N79C.

2. SPEC-C27S, N79C

To introduce the coupling point for antigen. This position was chosen from the crystal structure of SPE-C to be well exposed and to not interfere with MHC class II binding.

3. SPEC-C27S, N79C, Y15A

To destroy TcR binding

4. SPEC-C27S, N79C, R181Q

To further limit binding of T cell Receptors.

| | |
|---|---|
| PCR overlap | |
| 1^{st} round—amplification in separate tubes preoduces two overlapping products. | |
| (+ indicates the position of the mutation to be introduced. | |
| ==== represents vector sequence | |
| ------- Represents target sequence) | |
| 5′ utility upper mutant primer | |
| -------→ -----+----→ | |
| ========----------------------------------------------------------------------------------------------------------------=========== | |
| ←---+------ ←--------- | |
| lower mutant primer | lower utility |

```
2^{nd} round=combines the products of the first amplifications and amplifies with the utility primers
5′ utility
-------→
========--------------------------------------------------+------
========--------------------------------------------------+------
                                                                                                          ------+-------------------------------------------------------------============
                                                                                                          ------|-------------------------------------------------------------============
                                                                                                                                                                                                                                                            ←
#-----------
#lower utility
Final product
========-------------------------------------------------+-------------------------------------------------------------============
========-------------------------------------------------+-------------------------------------------------------------============
            Product is subcloned into the expression vector.
```

Glutathione-Agarose was manufactured according to previously published methods^{22,23}. Recombinant SPE-C protein was purified after cleavage of the fusion protein with trypsin by ion cation exchange chromatography according to the method described in reference 5 which is incorporated herein. Purified SPE-C was crystalised and the 3-D structure determined according to Roussel, 1997 (Ref 26), which is incorporated herein by reference.

Identification of amino acids in SPE-C that are important to TcR binding were determined by a combination of molecular modelling of the 3D crystal structure of SPE-C and comparison with known TcR binding residues of the related superantigen SEB.

### Rational Mutagenesis of Residues thought to be part of the TcR Interface

TcR binding residues were targeted by site-directed mutagenesis using the method of PCR overlap²⁴. The synthetic primers used to produce each mutation are described in the accompanying table of primers (Table 1). The process of introducing two mutations was performed sequentially as described in the accompanying diagrams describing the sequential introduction of successive mutations in SPE-C and the method of PCR overlap which is used to introduce said mutations.

The mutant form of SPE-C of the present invention was confirmed by automated DNA sequencing (Licor Inc. USA) then inserted into the pGEX expression vector between the BamH1 and EcoR1 restrictions sites according to the manufacturers description of the cloning site for this vector. A strain of E. coli DH5a was transformed with the recombinant vector and colonies expressing the pGEX fusion protein were isolated to grow up in large scale cultures for the purposes of protein purification.

To test the effects of mutations in the TcR binding site, recombinant proteins were added to cultures of human peripheral blood lymphocytes, isolated by standard techniques (for examples of techniques see Handbook Of Experimental Immunology, ed. D. M. Weir, Blackwell Scientific Publications), to determine what concentration of recombinant SPE-C was required to stimulate the proliferation of human T cells. Wild-type type SPE-C normally stimulates human T cells at 50% of maximal proliferation at 0.2 pg/ml. Two residues were identified from these studies that when mutated, reduce T cell proliferation by 1,000,000 fold when compared to wild-type SPE-C. These residues are Y15 and R181. SPE-C molecules with these two mutations (SPEC-Y15A, R181Q) no longer stimulate human T cells

Amino acid residues in superantigens that are important to the interaction with T cell Receptor have been identified from the present mutational studies and those of others (Table 2 below). Loss of T cell activation is determined by in vitro T cell proliferation assays (see below) and compared to the activity of wild-type molecule. All mutants are also assessed for their ability to bind to MHC class II by a number of assays including direct binding to MHC class II expressing B cells as well as Biacore studies with soluble forms of both superantigen mutant and MHC class II.

3D crystal structures of the superantigens SEC3 bound to a murine T cell Receptor ^{4,13} provides the most complete information about the nature of superantigen/TcR interaction but is limited to those with SEC3-like activity. Most single point mutations result in only a small loss in superantigen activity due to only small reductions in binding affinity to the TcR. It is rare to find a single mutation that completely abrogates all mitogenic potential. Only SPE-C Y15A has been shown (Yamoaka et al, Infect. Immunol. 1998 66:5020 and McCormick et al, J. Immunol. 2000 165: 2306-2312) to cause more than a 1000-fold reduction in T cell responses to a superantigen.

The combined mutations producing SPE-C Y15A, R181Q of the present invention generates a form of SPE-C that has no detectable T cell activating potential.

By homology modelling of the 3D crystal structures of other SAgs important regions for binding to the TcR can be identified and corresponding mutants prepared and used to generate immunomodulators of the present invention.

### Example 3

### T Cell Proliferation assay

The T cell proliferation assay used was a standard technique described for example in REF 5, incorporated herein by reference Purified recombinant mutant superantigens are incubated with freshly isolated human peripheral blood lymphocytes at varying dilutions in microtitre plates for 3 days. A fixed amount of ³H thymidine is added on the 3^{rd} day and the cells are harvested on day 4. The amount of ³H thymidine incorporated into the cellular DNA is measured by scintillation autography and is a direct measure of the degree of cell proliferation. Mutant superantigens are compared to wild-type superantigens. The proliferative potential of a given superantigen or mutant is expressed as the concentration required to induce 50% of its maximal stimulation (P₅₀%).

A fully ablated TcR binding negative superantigen is defined herein as one that displays less than about 0.0001% of proliferative activity of the wild-type superantigen (i.e. a 1 million-fold reduction in activity).

TABLE 2
Amino acid residues implicated in
TcR binding of known superantigens.

| | Residues implicated in TcR binding sites | References |
|---|---|---|
| SEA | N25, P206, D207 | 5, 14 |
| SEB | N23, Y90 | 12 |
| SEC3 | G19, T20, N23, Y26, N60, Y90, V91, G102, | 13, 4 |
| | K103, V104, G106, F176, Q210 | |
| SEE | N23, S206, N207 | 5, 14 |
| TSST | Tyr115, Glu132, His135, Ile140, His141 and | 15, 16 |
| | Tyr144, Q136A | |
| SPE-C | Y15*, R181* | Present |
| | | invention |
| SMEZ-2 | D42N, W75L, Y77A, K182Q, S7A, N11A, | Present |
| | D181A | invention |

Those in bold indicate mutations that decrease activity by more than 100-fold.
*Mutation that totally ablates T cell responses

Primary DNA sequences of the wild-type and the mutant form of SPE-C are detailed below:

SPE-C wild type (from GenBank)

Streptococcus pyogenes pyrogenic exotoxin C gene, 5′ end cds

[image]

### Protein Sequence (Combined Mutants)

DSKKDISNVK SDLLAAYTIT PYDYKDSRVN FSTTHTLNID
TQKYRGKDYY ISSEMSYEAS QKFKRDDHVD VFGLFYILCS
HTGEYIYGGI TPAQNNKVNH KLLGNLFISG ESQQNLNNKI
ILEKDIVTFQ EIDFKIRKYL MDNYKIYDAT SPYVSGRIEI
GTKDGKHEQI DLFDSPNEGT QSDIFAKYKD NRIINMKNFS
HFDIYLE

### Example 4

### Purification of Recombinant Wild-type and Mutant Proteins

Recombinant wild-type or mutant superantigens are expressed in E. coli. Two commercial vectors pGEX-2T (Pharmacia) and pET32A (New England Biolab) have been modified to introduce a new proteolytic cleavage site between the fusion protein and the superantigen. Separation of the two halves of the fusion protein is accomplished with the highly specific 3C protease that only cleaves at the single recognition site. Two methods are currently used to purify fusion proteins.

a. pGEX-2T produces a fusion protein with the N-terminal component as the Glutathione S-Transferase linked to the superantigen sequence through a protein linker that contains a 3C-protease cleavage site. The fusion protein is purified from the crude bacterial lysate in single step purification on glutathione agarose. Fusion protein is eluted from the glutathione agarose with a buffer containing 5 mM glutathione and cleaved by the addition of recombinant 3C protease. Superantigen is further purified by ion exchange HPLC chromatography.

b. pET32-A-3C. Protein is expressed as a stable thioredoxin fusion protein with a 6 histidine tag allowing single-step purification by metal chelation chromatography. Separation of the thioredoxin from superantigen is achieved by cleavage with recombinant 3C protease followed by HPLC ion exchange chromatography.

### Expression and Purification of the Recombinant Protein

E.coli transformants are grown overnight at 37° C. in a small 100 ml starter culture of Luria Broth (LB) containing 50 mg/ml ampicillin. A 1 liter culture is seeded in the morning and grown to mid-log phase, when IPTG is added to 0.1 mM to induce expression of the fusion protein. The culture is continued for 3 hours at which time cells are pelleted by centrifugation and disrupted by a combination of lysozyme and sonication.

The clarified lysate is passed over either a 5 ml GSH agarose column or a Ni-NTA column. After thorough washing, bound protein is eluted by either 5 mM GSH (GSH agarose) or a buffer containing imidazole (MC chromatography).

The fusion protein is cleaved overnight at room temperature by recombinant 3C protease at a ratio of 1:500 (i.e. 2 mg 3C protease to 1 mg fusion protein). Superantigen is separated from fusion protein by two rounds of cation exchange chromatography. Protein is filter sterilised and stored at 1 mg/ml at 4° C. until required.

### Introduction of Disulphide Coupling Sites into SPE-C

An exposed cysteine residue has been introduced into the N-terminus of a TcR negative SPE-C at position N79. N79 is located within the putative TcR binding site. Several positions were tested before a residue was identified that met the following criteria

a. Surface exposed and accessible

b. Displayed efficient coupling of synthetic peptide

c. Did not interfere with MHC class II binding

d. Did not render the resulting SAG:peptide conjugate insoluble.

In addition to the introduced cysteine, a naturally occurring cysteine residue at position 27 was mutated to serine to avoid complications with refolding and interference with coupling.

The mutant of SPE-C used herein to provide examples of in vitro and in vivo immunomodluatory activity is SPEC-Y15A.C27S.N79C.R181Q, which is a composite of all mutations so far described above that abrogates TcR binding (Y15A and R181Q), introduce an efficient coupling residue (N79C) and removes a naturally occuring cysteine which interfered with coupling (C27S)

### Example 5

### A Truncated Version SPEC Lacking the N-terminal Domain

In addition to the SPEC- SPEC-Y15A.C27S.N79C, an SPEC truncated mutant has been developed by deleting residues 22-90 (SPEC(-20-90))from the wild-type sequence This removes the entire TcR binding region plus the small N-terminal domain.

This truncated mutant expresses very well in E. coli, is soluble and retains MHC class II binding activity. A cysteine residue has been introduced at position 92 to effect antigen coupling using the same method as described for the full length SPEC-Y15A.C27S.N79C molecule. The importance of this mutant is that it is much smaller, less antigenic (less likely to promote anti-SPEC antibody responses), and will be entirely devoid of any TcR binding ability. It is most unlikely that this truncated SPEC will have any toxicity effects in vivo that are normally associated with wild-type toxins.

The primary nucleotide sequence of truncated version of SPE-C is detailed below:

[image]

### Example 6

### TcR Binding Defective Versions of SMEZ and SEA

In addition to SPE-C, TcR binding mutants of both SMEZ and SEA using site directed mutagenesis have been prepared. Comparative data of mutant vs wild-types on T cell proliferation is presented in table 3.

TABLE 3
SMEZ mutants defective in TcR binding

| Mutant | P50% (pg/ml) | Reduction |
|---|---|---|
| SMEZ-2 wild type | 2.0 | |
| SMEZ-2 W75L | >10 ng/ml | >100,000 |
| SMEZ-2 D42N | 10 ng/ml | 10,000 |
| SMEZ-2 | >10 ng/ml | >100,000 |
| W75L.D42N.K182Q | | |

The aim was to produce mutants which stimulate T cells at, for example, about 0.0001% of the activity of the wild type SAG. In addition, a cysteine residues is introduced in the same position relative to N79 in SPE-C.

Including two other superantigens is important to determine whether enhancement of immunogenicity is a feature of all superantigens, or specific to SPE-C. It is clearly broadly applicable, using the principles and techniques described herein.

Similar truncation mutants can be made for other superantigens such as SEA and SMEZ, using the methodology employed for the SPE-C mutants and the information on the Tcell receptor binding regions of the SAGs already published (for example reference #4, incorporated herein by reference).

### Example 7

### Peptide Coupling Procedure

Both protein and peptide are stored in 10 mM phosphate pH6.0 under nitrogen to prevent oxidation and auto-dimerisation through the free cysteine.

Synthetic peptide containing a C-terminal cysteine residue and SPEC-Y15A.C27S.N79C are mixed together and incubated at room temperature for 1 hour at a molar ratio of 1:2 in a alkaline buffer containing 1 μM Cu²⁺. The copper acts as a redox catalyst. In the example below, a synthetic peptide of the pigeon cytochrome C (PCC) is provided, but this method will work for other peptides also so long as a free sulphur atom is present in the peptide.

| SPEC- | | |
|---|---|---|
| Y15A.C27S.N79C.R181Q | PCC peptide | |
| (MW 26,500) | (RADLIAYLKQATKC) | |
| 10 mg/ml | (MW 1400) 10 mg/ml | |
| (380 mM) | (700 mM) | Buffer |
| 100 μl | 10 μl | 200 mM Tris |
| | | pH8.0, 1 μM |
| | | CuSO₄ |

Routinely >80% of SPEC-Y15A.C27S.N79C.R181 is shown to couple to peptide in a ratio of 1:1 Efficiency of coupling is assessed by SDS polyacrylamide gel electrophoresis. The SPEC-Y15A.C27S.N79C:peptide conjugate has a slower mobility on SDS PAGE consistent with an increase in molecular weight from the addition of a single peptide. Addition of 1 mM dithiothreitol (DTT) to the conjugate prior to SDS PAGE increases the electrophoretic mobility consistent with a reduction in molecular weight. This indicates that peptide coupling is via a reversible disulphide bond formation—a feature deemed important for dissociation of peptide once inside the APC.

### Example 8

### Testing of Responses to SAG:Peptide Conjugates

### The 5C.C7 T cell Receptor Transgenic Mouse

This mouse was obtained from The Malaghan Institute for Medical Research, Wellington School of Medicine, Mein St Wellington South, New Zealand

These mice were first generated by Berg et al (Ref 17).

The 5C.C7 transgenic mouse was originally constructed by Berg et al. ¹⁷. This mouse is transgenic for a TcR specific for the pigeon cytochrome C (PCC) peptide presented by mouse I-A^{d}. Greater than 80% of mature T cells from 5C.C7 mice express the transgenic TcR and respond to synthetic PCC peptide RADLIAYLKQATK in vitro. This mouse provides an excellent means to test PCC specific T cell responses both in vitro and in vivo as well as conduct adoptive transfer experiments. Adoptive transfer is a powerful method that allows the introduction of PCC reactive T cells into non-transgenic mice to study responses at varying T cell precursor frequencies.

### Antigenicity of SAG:PCC Peptide to 5C.C7 T Cells

This experiment determines how potent the SAG:peptide conjugate is in vitro. It is a test of how well the antigen is taken up and presented by the APCs present in culture and whether the binding of SAG to MHC class II enhances presentation to T cells.

Lymph node T cells from adult 5C.C7 mice were incubated with varying amounts of either synthetic PCC peptide alone, SPEC-Y15A.C27S.N79C, PCC peptide and SPEC-Y15A.C27S.N79C.R181 unconjugated or conjugated prior to addition in culture. MHC class II restricted T cell responses were measured by a 3-day ³H thymidine incorporation assay. Methods used were standard techniques such as those described Current Protocols in Immunology (1998) Colligan, J., Kuisbeck, A. M. Shevach, E. M. and W. Strober eds. John Wiley & Sons, Inc (ref 25)

### Results

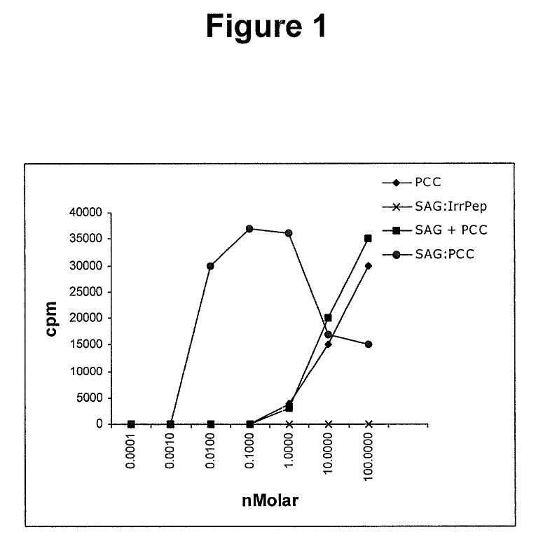

FIG. 1 indicates that 5C.C7 T cells responded to 10,000 times less SAG:PCC conjugate than the peptide alone. Optimal response to the SAG:PCC conjugate occurred at 10 pM compared to 100 nM for the same components added in unconjugated form. No response was observed to SAG: irrelevant peptide indicating that the response was specific to the PCC peptide.

### Immunogenicity of SAG:PCC conjugate in 5C.C7 Mice

This tests the ability of the SAG:peptide conjugate to generate an immune response in vivo and is a test of it's immunogenicity—that is to stimulate and expand peptide specific T cells.

(i) Adoptive transfer of 5C.C7 T cells into wild-type C57B1/6 mice Normal female C57B1/6 recipient mice receive 5×10⁶5C.C7 lymph node cells IP 1 week prior to immunisation.

### Immunisation Protocol

Antigens were injected as a single subcutaneously (SC) dose as a stable emulsion with Freund's incomplete adjuvant in mature female C57B1/6 mice that had previously received 5C.C7 T cells. Two mice were injected for each dose with one of:

1. PCC peptide alone (1 and 100 mg)

2. PCC peptide +SPEC-Y15A.C27S.N79C.R181

3. SPEC:PCC conjugate (20 ng) Mice were sacrificed 10 days later and the draining mesenteric lymph nodes removed. 1×10⁵ lymph node cells/well were cultured in duplicate with varying amounts of synthetic PCC peptide and the proliferative response of T cells measured by the 3 day ³H thymidine incorporation assay.

### Results

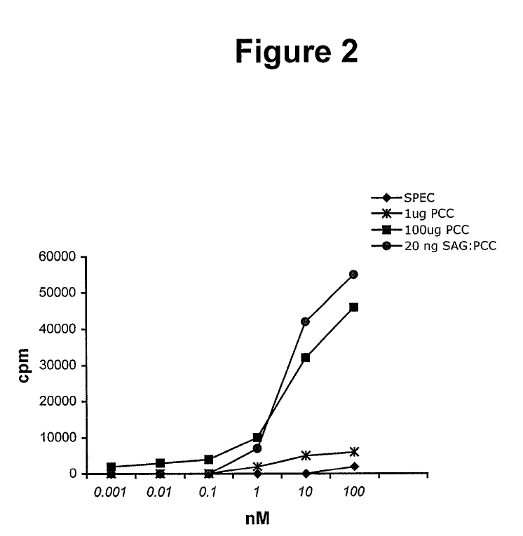

FIG. 2 indicates that the lowest dose of SAG:PCC conjugate used to immunised 5C.C7 mouse was 20 ng and this produced optimal immunity equivalent to 100 mg of free PCC peptide. 1 mg of PCC peptide was non immunogenic. Thus the SAG:PCC conjugate was at least 10,000 times more immunogenic than free peptide. Irrelevant peptides coupled to SPEC generated no detectable immune response. It is likely that even lower doses of SAG:PCC conjugate will be immunogenic, increasing the effective difference in potency between conjugated and unconjugated PCC peptide to 100,000 times.

These studies show that SPEC-Y15A.C27S.N79C.R181 acts as an efficient delivery vehicle for poorly immunogenic antigens such as synthetic peptides. Not only is the peptide significantly more antigenic in vitro, but this also translates into enhanced immunogenicity in vivo. The immunogenicity of the PCC peptide increased by at least 10,000 times by coupling to the TcR binding defective superantigen SPEC-Y 15A.C27S.N79C.

SPE-C mutant defective in MHC class II binding does not enhance antigenicity of the PCC peptide.

A recombinant mutant of SPE-C was created that disrupts the single zinc binding site to MHC class II. This mutant was coupled to synthetic PCC peptide and tested for its ability to stimulate 5C.C7 T cells in vitro compared to normal SPEC:PCC conjugate.

The results show that the mutant SPEC:PCC conjugate was no more antigenic than the SPEC+free peptide alone. This indicates that enhanced antigenicity is a result of SPE-C's ability to bind to cells expressing MHC class II, a function unique to superantigens.

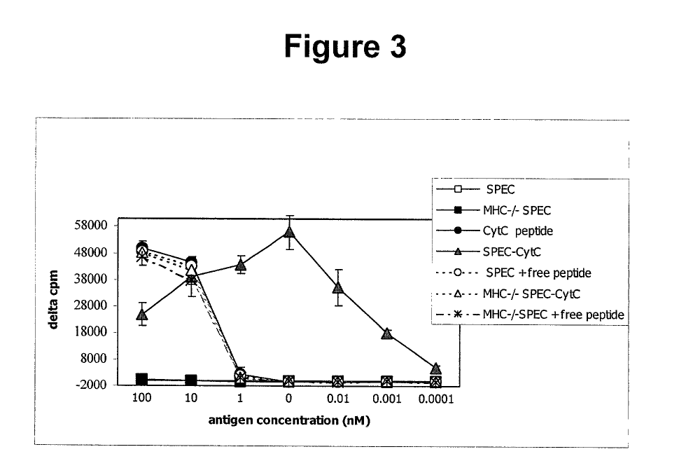

FIG. 3 shows data which reveals the importance of MHC class II binding to enhancement of antigenicity and that SPEC is not simply acting as a “non-specific” carrier protein.

### Example 10

### Coupling of Multiple Peptides

Coupling need not be limited to individual peptides. Because immune responses to peptides are tightly restricted by the MHC polymorphisms of the host, it might be appropriate in some circumstances, to immunise with sets of peptides to generate broad spectrum immunomodulatory agents. Multiple peptides representing various components of a larger antigen such as a virus, bacteria or other protein antigen may be coupled by procedures described above or modified versions therefore which would be clear to those skilled in the art, to provide a mixed peptide:SAG conjugate antigen response to increase the diversity of the conjugate. Moreover, the ratio of peptides could be easily controlled to fine tune the immune response to a more desired outcome.

In further embodiments of the present invention, and applying the principles described herein, the following can also be accomplished:

MHC class I and class II restricted peptides may be combined to provide improved helper CD4 and cytolytic CD8 effector cells.

Immunodominant peptides from more than one viral antigen may be combined to promote selective anti-viral immunity.

Peptides from regions of viral antigens that do not normally predominate in the protective immune response but represent regions of the virus essential to its replication or life cycle and are by nature strongly conserved may be used. This is particularly important in developing vaccines against highly mutating viruses such as retroviruses (e.g. HIV).

Peptides and other antigens can be combined together and delivered by the immunomodulators to enhance or modulate the immune response.

### Example 11

### Coupling of Larger Antigens and Complex Structures

Polypeptides and proteins can be coupled using the same procedures described above by reversible disulphide interchange to mutant SAGs. In addition, larger structures such as viruses can be “coated” with a TcR defective SAG by first treating the virus with a chemical that introduces a reactive sulphydryl group.

If the polypeptide has a naturally occurring exposed cysteine residues, coupling may be achieved to SAG directly without the need to introduce a reactive sulphydryl group. In this case, coupling would follow the established procedure outlined above.

### Chemical Coupling Methods

If the polypeptide does not have a naturally occurring cysteine, there are two methods that introduced a reactive sulphydryl group

a. A cysteine residue can be introduced genetically into the recombinant peptide and the polypeptide expressed from a heterologous expression system (prokaryotic or eukaryotic)

b. A chemical coupling reagent can be employed to introduce a reactive sulphydryl into the target protein or larger structure. A number of chemicals can be employed to introduce reactive sulphur groups onto proteins and other structures. One such chemical is N-succinimidyl S-acetylthiolproprionate (SATA—Piece Chemicals) and its close analogue SATP. This chemical converts a free amino groups on a protein or larger structure to a protected sulphydryl group which is activated with hydroxylamine. This allows coupling of other sulphydryl containing proteins such as SPEC-Y15A.C27S.N79C.R181 via a reducible disulphide bond.

Relevant techniques are described in Ref. 21, incorporated herein by reference.

Delivery of proteins known to generate protective immunity for a particular pathogen can be made more immunogenic by first conjugating the protein to a TcR ablated SAG. The polypeptide would be broken down internally by the APC to present multiple restricted peptide epitopes to the host immune system. Anti-viral immunity might be enhanced by adding on molecules that selectively target the virus to APCs such as dendritic cells.

### Example 12

### Multiple Sclerosis and EAE in Mice

For multiple sclerosis, the predominant self antigen appears to be the Myelin Basic Protein (MBP) which is the major component of the myelin sheath. Experimental Allergic Encephalitis (EAE) is a well-established mouse model for the human disease multiple sclerosis. EAE can be generated by immunising susceptible mice with myelin basic protein (MBP) which produces anti-MBP reactive T cells that attack the myelin coating of nerves, leading to the encephalitic disease characterised by loss of motor control ¹⁸.

The EAE model can be used to examine the ability of mutant SAG:MBP peptides or mutant SAG:MBP protein conjugates to inhibit the start of the disease, or to suppress existing disease ¹⁹. Peptides (both agonist and antagonist) from the myelin basic protein (MBP) will be tested for their ability to suppress the onset of the EAE disease in mice.

### Example 13

### Anti-viral Responses and MHC Class I Restricted Peptides

Mutant SAG:peptide conjugates could also serve to enhance MHC class I restricted CTL responses. CD8 positive CTL recognise peptides presented by MHC class I derived from viral infection and replication via the endogenous processing pathway. It has been shown however that there is significant cross-talk between the endogenous and exogenous pathway for peptides to be “shared” by both MHC class I and MHC class II molecules.

Protective cytolytic responses against viral infection or tumours are believed to require an obligate CD4 MHC class II dependent response as well as MHC class I restricted CD8 responses to provide long lasting protective immunity. Thus vaccines constructed from the conjugation of MHC class I restricted peptides and SAG mutants or a combination of both MHC class I and MHC class II restricted peptides would offer a flexible approach to designing efficient vaccines which promote both CD4 and CD8 responses

### The LCMV₃₃₋₄₁ peptide and the 318 Transgenic Mice

The 318 transgenic mouse is a C57BL/6 mouse with a transgenic TcR which recognises the lymphocyte choriomeningitis virus (LCMV) peptide in the context of the MHC class I antigen H-2D^{b 20}. The sequence of the active peptide is CKAVYNFATM which originates from the nucleocapsid protein. The 318 mouse will be used to model the ability of SPEC-Y15A.C27S.N79C.R181 and other TcR defective SAGs to deliver MHC class I restricted peptides to CD8 cytotoxic T cells. Efficiency of delivery will be measured by the amount of SAG:LCMV conjugate required to generate a cytotoxic response against target cells pre-incubated with LCMV peptide (standard cytotoxic assay).

### The ⁵¹Cr Release Cytotoxicity Assay to Measure MHC Class I Restricted Responses

Target cells (P814) are incubated with ⁵Cr and pulsed with LCMV peptide for 1 hour at 37° C. Cells are washed by centrifugation and mixed with lymph node cells from immunise mice at varying E:T ratios.

Cells are centrifuged lightly and incubated at 37° C. for 1 hour. Supernatant is removed and counted for ⁵¹Cr to determine the degree of cell lysis. Synthetic LCMV peptide modified at position 8 (M8C) will be coupled to SPEC-Y15A.C27S.N79C.R181using the same method as described above. SAG:LCMV will be used to determine the in vitro response in lymph node cells from 318 mice.

### Resistance to Viral Infection

Mice infected with LCMV succumb within 14 days to the cytopathic effects. Mice immunised against LCMV develop a CTL response which provides full protection against. Mice immunised with SAG:LCMV will be tested for their resistance to wild-type LCMV virus.

### Example 14

### Anti-tumour Immunity

Many novel cancer immunotherapies attempt to break host tumour tolerance by targeting potential tumour specific antigens (usually lineage specific or differentiation antigens) directly to dendritic cells. We will test the hypothesis that TcR defective SAGs might usefully target tumour specific antigens to APCs and promote costimulatory signals that enhance antigen presentation. Initial studies will employ a tumour model in the 318 TcR transgenic mouse.

### The Lewis Lung Carcinoma and the 318 Transgenic Mouse

We will initially employ a mouse model of tumour protection using the 318 transgenic mouse. A Lewis Lung carcinoma cell line transfected with a gene expressing the LCMV glycoprotein provides a model to investigate the ability of 318 mice to reject tumours. This cell line has high metastatic potential.

Mice will be immunised with SAG:LCMV peptide and then inoculated with tumour cells. The degree of metastatic foci will be established at varying time points following inoculation and compared with non-immunised mice.

Mice will also be inoculated and then immunised at varying time points following tumour inoculation to determine whether immunisation protects established tumour growth.

### Example 15

### Increasing the Antigenicity of a Whole Protein to T Cells by Coupling to SAG

1 mg whole Pigeon Cytochrome C protein (PCC) (Sigma) was treated with 1 mg of the cross-linked reagent N-succinimidyl S-acetylthioproprionate (SATP)(Pierce) for 1 hour at room temperature at pH7.0. Excess cross-linker was removed by gel chromatography using well established protocols, and the PCC-SATP activated with 0.1M hydroxylamine and incubated with 100 μg recombinant SAG for 1 hour at pH8.5 to allow the proteins to couple. Conjugate was separated from free reactants by size exclusion chromatography according to well established protocols.

This method results in approximately 30% of the SAG forming a conjugate with PCC in a molar ratio of 1:1.

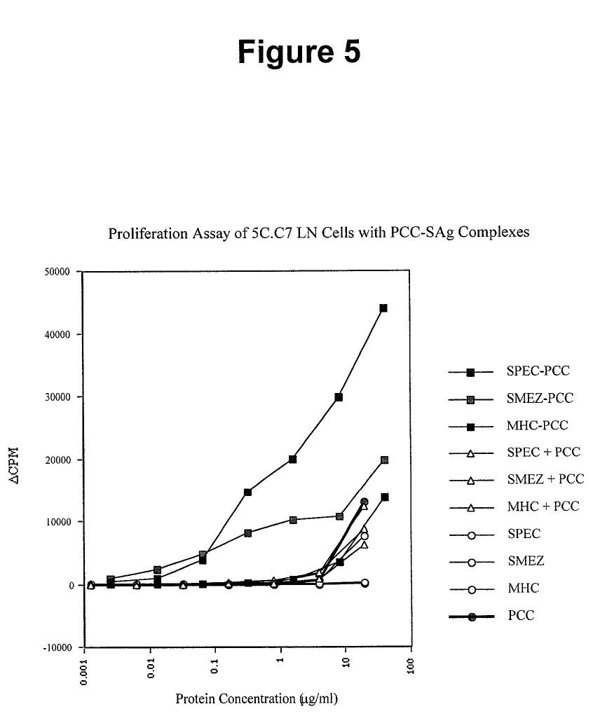

Conjugates were incubated with cultures of lymph node cells from T cells 5C-C7 mice and proliferation of T cells measured by ³H thymidine incorporation after 3 days, according to well established protocols. Results of these studies are shown in FIG. 5.

The results show a substantial increase in the antigenicity towards PCC protein when conjugated to either SPEC or SMEZ. They further emphasises the importance of binding of the SAG to MHC class II to achieve increased antigenicity.

Some of the advantages and features of the exemplary TcR defective immunomodulatory conjugates of the present invention are the following:

a. The SAG is totally defective in binding to all TcRs and thus will be non-toxic in vivo.

b. Coupling of peptides is simple, efficient and reversible and broadly applicable.

c. The SAG:peptide conjugate is soluble.

d. SAG binding to MHC class II enhances APC activation of immunogenic and non-immunogenic moieties.

Although the present invention has been described with reference to certain preferred embodiments it will be understood that variations, which are in keeping with the broad principles and the spirit of the invention, are also contemplated to be within its scope.

### References

A. Staphylococcal Superantigens

SEA Betley M J, Mekalanos J J (1988). Nucleotide sequence of the type A staphylococcal enterotoxin gene. J Bacteriol 170(1):34-41. Huang I Y, Hughes J L, Bergdoll M S, Schantz E J (1987). Complete amino acid sequence of staphylococcal enterotoxin A. J Biol Chem. 262(15):7006-13.

SEB Jones C L, Khan S A (1986). Nucleotide sequence of the enterotoxin B gene from Staphylococcus aureus. J Bacteriol. 166(1):29-33.

SEC1 Bohach G A, Schlievert P M (1987). Nucleotide sequence of the staphylococcal enterotoxin C1 gene and relatedness to other pyrogenic toxins. Mol Gen Genet. 209(1):15-20.

SEC2 Bohach G A, Schlievert P M (1989). Conservation of the biologically active portions of staphylococcal enterotoxins C1 and C2. Infect Immun. 57(7):2249-52.

SEC3 Hovde C J, Hackett S P, Bohach G A (1990). Nucleotide sequence of the staphylococcal enterotoxin C3 gene: sequence comparison of all three type C staphylococcal enterotoxins. Mol Gen Genet. 220(2):329-33.

SED Bayles K W, Iandolo J J. 1989. Genetic and molecular analyses of the gene encoding staphylococcal enterotoxin D. J Bacteriol. 171(9):4799-806.

SEE Couch J L, Soltis M T, Betley M J (1988). Cloning and nucleotide sequence of the type E staphylococcal enterotoxin gene. J Bacteriol. 170(7):2954-60.

SEG Munson S H, Tremaine M T, Betley M J, Welch R A. 1998. Identification and characterization of staphylococcal enterotoxin types G and I from Staphylococcus aureus. Infect Immun. 66(7):3337-48.

SEH Ren K, Bannan J D, Pancholi V, Cheung A L, Robbins J C, Fischetti V A, Zabriskie J B. 1994. Characterization and biological properties of a new staphylococcal exotoxin. J Exp Med. 180(5):1675-83.

SEI Munson S H, Tremaine M T, Betley M J, Welch R A. 1998. Identification and characterization of staphylococcal enterotoxin types G and I from Staphylococcus aureus. Infect Immun. 66(7):3337-48.

SEJ Zhang, S., Iandolo, J. J. and Stewart, G. C. 1998. The enterotoxin D plasmid of Staphylococcus aureus encodes a second enterotoxin determinant (sej). FEMS Microbiol. Letters 168; 227-233.

TSST Blomster-Hautamaa D A, Kreiswirth B N, Kornblum J S, Novick R P, Schlievert P M. 1989. The nucleotide and partial amino acid sequence of toxic shock syndrome toxin-1. J Biol Chem. 261(33):15783-6.

B. Streptococcal Superantigens

SpeA Johnson L P, L'Italien J J, Schlievert P M. 1986. Streptococcal pyrogenic exotoxin type A (scarlet fever toxin) is related to Staphylococcus aureus enterotoxin B. Mol Gen Genet. 203(2):354-6.

SpeB Hauser A R, Schlievert P M. 1990. Nucleotide sequence of the streptococcal pyrogenic exotoxin type B gene and relationship between the toxin and the streptococcal proteinase precursor. J Bacteriol. 172(8):4536-42.

SpeC Goshorn S C, Schlievert P M. 1988. Nucleotide sequence of streptococcal pyrogenic exotoxin type C. Infect Immun. 56(9):2518-20.

SpeF Norrby-Teglund A, Newton D, Kotb M, Holm S E, Norgren M. 1994. Superantigenic properties of the group A streptococcal exotoxin SpeF (MF). Infect Immun. 62(12):5227-33.

SpeG Proft T, Moffatt S L, Berkahn C J, Fraser J D. 1999. Identification and characterization of novel superantigens from Streptococcus pyogenes. J Exp Med. 189(1):89-102.

SpeH Proft T, Moffatt S L, Berkahn C J, Fraser J D. 1999. Identification and characterization of novel superantigens from Streptococcus pyogenes. J Exp Med. 189(1):89-102.

SpeI McLaughlin R. L., Sezate, S., Ferretti J. J. 1999. Molecular Characterization of Genes Encoding SPE-H and SPE-I. XIV. LISSSD.

SpeJ Proft T, Moffatt S L, Berkahn C J, Fraser J D. 1999. Identification and characterization of novel superantigens from Streptococcus pyogenes. J Exp Med. 189(1):89-102.

SSA Mollick J A, Miller G G, Musser J M, Cook R G, Grossman D, Rich R R. 1993. A novel superantigen isolated from pathogenic strains of Streptococcus pyogenes with aminoterminal homology to staphylococcal enterotoxins B and C. J Clin Invest. 92(2):710-9.

SMEZ Kamezawa Y, Nakahara T, Nakano S, Abe Y, Nozaki-Renard J, Isono T. 1997. Streptococcal mitogenic exotoxin Z, a novel acidic superantigenic toxin produced by a T1 strain of Streptococcus pyogenes. Infect Immun. September;65(9):3828-33.

SMEZ-2 Proft T, Moffatt S L, Berkahn C J, Fraser J D. 1999. Identification and characterization of novel superantigens from Streptococcus pyogenes. J Exp Med. January 4;189(1):89-102.

SMEZ-3-SMEZ-24 Proft T, Moffatt S L, Weller K D, Paterson A, Martin D, Fraser J D. 2000. The streptococcal superantigen SMEZ exhibits wide allelic variation, mosaic structure, and significant antigenic variation. J Exp Med. 15;191(10):1765-76.

### General References

1. Kotzin, B. L., Leung, D. Y., Kappler, J. & Marrack, P. Superantigens and their potential role in human disease. Adv Immunol 54, 99-166 (1993).

2. Marrack, P. & Kappler, J. The Staphylococcal enterotoxins and their relatives. Science 248, 705-711 (1990).

3. Fraser, J. D., Arcus, V., Kong, P., Baker, E. N. & Proft, T. P. Superantigens—powerful modifiers of the immune system. Molecular Medicine Today 6, 125-135 (2000).

4. Li, H., Llera, A. & Mariuzza, R. A. Structure-function studies of T-cell receptor-superantigen interactions. [Review] [52 refs]. Immunological Reviews 163, 177-86 (1998).

5. Hudson, K. R. et al. Staphylococcal enterotoxin A has two cooperative binding sites on major histocompatibility complex class II. J. Exp. Med. 182, 711-20 (1995).

6. Li, P. L., Tiedemann, R. E., Moffat, S. L. & Fraser, J. D. The superantigen streptococcal pyrogenic exotoxin C (SPE-C) exhibits a novel mode of action. Journal of Experimental Medicine 186, 375-83 (1997).

7. Jardetzky, T. S. et al. Three-dimensional structure of a human class II histocompatibility molecule complexed with superantigen. Nature 368, 711-8 Issn: 0028-0836 (1994).

8. Banchereau, J. et al. Immunobiology of dendritic cells [Review]. Annual Review of Immunology 18 (2000).

9. Banchereau, J. & Steinman, R. M. DENDRITIC CELLS AND THE CONTROL OF IMMUNITY [Review]. Nature 392, 245-252 (1998).

10. Tiedemann, R. E. & Fraser, J. D. Cross-linking of MHC class II molecules by staphylococcal enterotoxin A is essential for antigen-presenting cell and T cell activation. Journal of Immunology 157, 3958-66 (1996).

11. Mehindate, K. et al. Cross-Linking Of Major Histocompatibility Complex Class Ii Molecules By Staphylococcal Enterotoxin a Superantigen Is a Requirement For Inflammatory Cytokine Gene Expression. Journal of Experimental Medicine 182, 1573-1577 (1995).

12. Marrack, P., Blackman, M., Kushnir, E. & Kappler, J. The toxicity of staphylococcal enterotoxin B in mice is mediated by T cells. J Exp Med 171, 455-64 (1990).

13. Fields, B. A. et al. Crystal structure of a T-cell receptor beta-chain complexed with a superantigen [see comments]. Nature 384, 188-92 (1996).

14. Irwin, M. J., Hudson, K. R., Fraser, J. D. & Gascoigne, N. R. Enterotoxin residues determining T-cell receptor V beta binding specificity. Nature 359, 841-3 (1992).

15. Acharya, K. R. et al. Structural basis of superantigen action inferred from crystal structure of toxic-shock syndrome toxin-1. Nature 367, 94-7 (1994).

16. Earhart, C. A. et al. STRUCTURES OF FIVE MUTANTS OF TOXIC SHOCK SYNDROME TOXIN-1 WITH REDUCED BIOLOGICAL ACTIVITY. Biochemistry 37, 7194-7202 (1998).

17. Berg, L. J. et al. Expression of T-cell receptor alpha-chain genes in transgenic mice. Molecular & Cellular Biology 8, 5459-69 (1988).

18. Wucherpfennig, K. W. & Strominger, J. L. Molecular mimicry in T cell-mediated autoimmunity: viral peptides activate human T cell clones specific for myelin basic protein. Cell 80, 695-705 (1995).

19. Brocke, S. et al. Induction of relapsing paralysis in experimental autoimmune encephalomyelitis by bacterial superantigen. Nature 365, 642-4 (1993).

20. Pircher, H., Burki, K., Lang, R., Hengartner, H. & Zinkernagel, R. M. Tolerance induction in double specific T-cell receptor transgenic mice varies with antigen. Nature 342, 559-61 (1989).

21. Duncan, R. J. S., Weston, P. D., Wrigglesworth, R. (1983) A new reaent which may be used to introduce sulphydryl groups into proteins, and its use in the preparation of conjugates for immunoassay. Anal. Biochem. 132, 68-73.

22. Sundberg, J. and J. Porath (1974) Attachment of group-containing ligands to insolbule polymers by means of bifunctional oxiranes. J. of Chromatography 90, 87-98.

23. Simons, P. and D. L. Vnder Jagt (1977) Purification of Glutathione-S-Transferease from human liver by glutathione-affinity chromatography. Anal. Biochem. 82 334-34

24. Ho, S N, Hunt H D, Horton R M, Pullen J K, Pease L R (1989) Site directed mutagenesis by overlap ectension using the polymerase chain reaction. Gene 67:31-40.

25. Current Protocols in Immunology (1998) Coligan, J, Kruisbeck A. M., Margulies D. H., Shevach, E., and W. Strober. Eds John Wiley & Sons NY.

26. Roussel A, Baker H M, Fraser J D, Baker E N (1997) Crystal structure of the streptococcal superantigen SPE-C: dimerisation and zinc binding suggests a novel mode of interaction with MHC class II molecules NATURE STRUCTURAL BIOLOGY. 4(8): 635-643

What is claimed is:

1. Immunomodulator which comprises an antigen-presenting- cell (APC) targeting molecule coupled to an immunomodulatory antigen, wherein said APC-targeting molecule mimics a superantigen but does not include a fully functional T-cell receptor binding site.

2. Immunomodulator which comprises an antigen-presenting cell (APC) targeting molecule coupled to an immunomodulatory antigen, wherein said APC-targeting molecule is a molecule which is structurally a superantigen but for a disrupted T-cell receptor binding site such that the molecule has little or no ability to activate T-cells.

3. An immunomodulator according to claim 1 or claim 2, wherein the T-cell receptor binding site, or at least a part thereof, of the antigen-presenting- cell (APC) targeting molecule has been modified by substitution or addition.

4. An immunomodulator according to claim 1 or claim 2, wherein the T-cell binding site of the antigen-presenting cell (APC) targeting molecule has been deleted.

5. An immunomodulator according to any one of claims 1 to 3, wherein the antigen-presenting cell (APC) targeting molecule is derived from Staphylococcus aureus and/or Streptococcus pyogenes.

6. An immunomodulator according to claim 5, wherein antigen-presenting cell (APC) targeting molecule is derived from SPE-C, SMEZ and/or SEA.

7. An immunomodulator according to claim 6, wherein the antigen-presenting cell (APC) targeting molecule is designated SPEC-Y15A as herein defined.

8. An immunomodulator according to claim 6 or claim 7, wherein the antigen-presenting cell (APC) targeting molecule is designated SPEC-Y15A R181Q.

9. An immunomodulator according to any one of claims 6 to 8, wherein the antigen-presenting cell (APC) targeting molecule is designated SPEC-Y15A.C27S.N79C.R181Q.

10. An immunomodulator according to any one of claims 1 to 9, wherein the antigen-presenting-cell (APC) targeting molecule is coupled reversibly to an immunomodulatory antigen.

11. An immunomodulator according to any one of claims 1 to 10, wherein the immunomodulatory antigen is a protein, a polypeptide and/or a peptide.

12. An immunomodulator according to any one of claims 1 to 10, wherein the immunomodulatory antigen is a nucleic acid.

13. An immunomodulator according to any one of claims 1 to 12, wherein the immunomodulatory antigen is non-immunogenic when not coupled to the antigen-presenting cell (APC) targeting molecule.

14. An immunomodulator according to claim any one of claims 4 or 10 to 13, wherein the antigen-presenting cell (APC) targeting molecule is SPEC (-20-90).

15. Pharmaceutical composition comprising an immunomodulator according to any one of claims 1 to 14 and a pharmaceutically acceptable carrier, adjuvant, excipient and/or solvent.

16. Vaccine comprising an immunomodulator according to any one of claims 1 to 14.

17. Method of therapeutic or prophylactic treatment of a disorder which requires the induction or stimulation of the immune system, comprising the administration to a subject requiring such treatment of an immunomodulator according to any one of claims 1 to 14, of a pharmaceutical composition according to claim 15 or of a vaccine according to claim 16.

18. A method according to claim 17, wherein the disorder is selected from the group consisting of bacterial, viral, fungal or parasitic infection, autoimmunity, allergy and/or pre-neoplastic or neoplastic transformation.

19. Use of an immunomodulator according to any one of claims 1 to 14 for the preparation of a medicament for the therapeutic or prophylactic treatment of a disorder which requires the induction or stimulation of the immune system.

20. Use according to claim 19, wherein the disorder is selected from the group consisting of bacterial, viral, fungal or parasitic infection, autoimmunity, allergy and/or pre-neoplastic or neoplastic transformation.

21. Method of preparing an immunomodulator comprising the steps of:
(a) introducing a modification and/or a deletion into the T-cell binding site of an antigen-presenting cell (APC) targeting molecule which is structurally a superantigen, and
(b) coupling thereto and immunomodulatory antigen.

22. A method according to claim 21, wherein the antigen-presenting cell (APC) targeting molecule is selected from the group of SPE-C, SMEZ and SEA.

23. A method according to claim 21 or claim 22, wherein the antigen-presenting cell (APC) targeting molecule is SPE-CY15A R181Q

24. A method according to any one of claims 21 to 23, wherein the antigen-presenting cell (APC) targeting molecule is designated SPEC-Y15A.C27S.N79C.R181Q.

25. A method according to claim 21 or claim 22, wherein the antigen-presenting cell (APC) targeting molecule is SPEC (-20-90).

26. Method of increasing antigenicity of a compound, comprising the coupling of said compound to an antigen-presenting-cell (APC) targeting molecule, wherein said APC-targeting molecule mimics a superantigen but does not include a fully functional T-cell receptor binding site.

27. A method according to claim 26, wherein said APC-targeting molecule is a molecule which is structurally a superantigen but for a disrupted T-cell receptor binding site such that the molecule has little or no ability to activate T-cells.

28. A method according to claim 26, wherein the T-cell receptor binding site, or at least a part thereof, of the antigen-presenting-cell (APC) targeting molecule has been modified by substitution or addition.

29. A method according to claim 26, wherein the T-cell binding site of the antigen-presenting cell (APC) targeting molecule has been deleted.

30. A method according to any one of claims 26 to 29, wherein the antigen-presenting cell (APC) targeting molecule is derived from Staphylococcus aureus and/or Streptococcus pyogenes.

31. A method according to claim 30, wherein antigen-presenting cell (APC) targeting molecule is derived from SPE-C, SMEZ and/or SEA.

32. A method according to claim 31, wherein the antigen-presenting cell (APC) targeting molecule is designated SPEC-Y15A as herein defined.

33. A method according to claim 31, wherein the antigen-presenting cell (APC) targeting molecule is designated SPEC-Y15A R181Q.

34. A method according to claim 31, wherein the antigen-presenting cell (APC) targeting molecule is designated SPEC-Y15A.C27S.N79C.R181Q

35. A method according to claim 31, wherein the antigen-presenting cell (APC) targeting molecule is SPEC (-20-90).

36. A method according to any one of claims 26 to 29, wherein the antigen-presenting- cell (APC) targeting molecule is coupled reversibly to said compound.

37. A method according to any one of claims 26 to 29, wherein the compound is selected from the group consisting of a protein, a polypeptide and/or a peptide, a carbohydrate or a nucleic acid.

38. A method according to any one of claims 26 to 29, wherein the compound is non-immunogenic when not coupled to the antigen-presenting cell (APC) targeting molecule.
